(19) United States
(12) Patent Application Publication
(10) Pub. No.: US 2026/0335230 A1
(43) Pub. Date: Oct. 29, 2026

(54) AQUEOUS INJECTABLE COMPOSITIONS OF TRILACICLIB

(71) Applicant: DR. REDDY'S LABORATORIES LIMITED, Banjara Hills (IN)

(72) Inventors: Mukesh Bothra, Banjara Hills (IN); Kaushal K. Jain, Banjara Hills (IN); Kuldipgiri Rajendragiri Goswami, Banjara Hills (IN); Subramanian Narayanaswamy, Banjara Hills (IN)

(73) Assignee: DR. REDDY'S LABORATORIES LIMITED, Banjara Hills (IN)

(21) Appl. No.: 19/650,955

(22) Filed: Apr. 17, 2026

(30) Foreign Application Priority Data
Apr. 18, 2025 (IN) IN202541037738

(51) Int. Cl. A61K 9/08; A61K 9/00; A61K 31/519; A61K 47/12; A61K 47/26

(52) U.S. Cl. CPC A61K 9/08; A61K 9/0019; A61K 31/519; A61K 47/12; A61K 47/26

(57) ABSTRACT

The present disclosure relates to stable aqueous injectable compositions of trilaciclib or pharmaceutical acceptable salts, hydrates, solvates, polymorphs, and mixtures thereof. More particularly, the present disclosure relates to stable aqueous ready-to-dilute pharmaceutical compositions containing trilaciclib or pharmaceutical acceptable salts, hydrates, solvates, polymorphs, and mixtures thereof and an organic acid. The present disclosure further relates to processes for preparation of stable aqueous injectable compositions of trilaciclib.

### CROSS REFERENCE TO RELATED APPLICATION

This application takes priority from an Indian application number IN202541037738 filed on Apr. 18, 2025, the contents of which is incorporated herein by reference.

### FIELD OF INVENTION

The present invention relates to aqueous injectable compositions of trilaciclib or pharmaceutical acceptable salts, hydrates, solvates, polymorphs, and mixtures thereof.

### BACKGROUND OF THE INVENTION

Trilaciclib dihydrochloride is approved as CDK4 Kinase Inhibitor to decrease the incidence of chemotherapy-induced myelosuppression in adult patients when administered prior to a platinum/etoposide-containing regimen or topotecan-containing regimen for extensive-stage small cell lung cancer. Trilaciclib dihydrochloride is structurally represented as

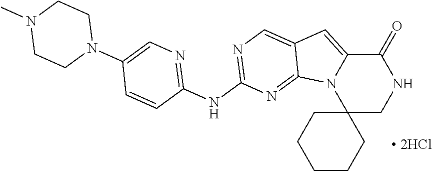 [SMILES: CN1CCN(c2ccc(Nc3ncc4cc5n(c4n3)C3(CCCCC3)CNC5=O)nc2)CC1]

Trilaciclib is available in the US market with the Brand Name COSELA® and marketed by G1 Therapeutics as a sterile, preservative free, lyophilized cake in a single dose vial for intravenous infusion after reconstitution and dilution. U.S. Pat. Nos. 8,598,186, 8,598,197, 9,957,276, 10,189,849, 10,189,850 & 10,927,120 describe trilaciclib and related compounds.

Each single dose vial of COSELA® contains the equivalent of 300 mg of trilaciclib (provided as 349 mg of trilaciclib dihydrochloride) and the following inactive ingredients: citric acid monohydrate (75.6 mg) and mannitol (300 mg), hydrochloric acid and sodium hydroxide to adjust pH. The lyophilized preparation needs reconstitution with 19.5 mL of 0.9% NaCl injection, USP or 5% dextrose injection, USP using a sterile syringe to obtain a concentration of 15 mg/mL of trilaciclib. Moreover, the re-constituted solution can be stored at 20° C. to 25° C. (68° F. to 77° F.) only for up to 4 hours prior to transfer to the infusion bag.

U.S. Pat. No. 9,487,530 discloses one example of pharmaceutical composition that includes hydrochloride salt of trilaciclib in an aqueous system for intravenous use, wherein the aqueous solvent system comprises of hydroxypropyl-beta-cyclodextrin and dextrose along with hydrochloric acid/sodium hydroxide as pH adjusting agents.

U.S. Pat. No. 10,988,479 discloses advantageous morphic forms of trilaciclib free base that can be delivered as an IV solution from lyophilized formulations. Additionally, it discloses that trilaciclib has less solubility in water and further less soluble as the pH increases to 7.2-7.4. Further discloses the solubility studies conducted for dihydrochloride salt form of trilaciclib in various solvent systems. While, a thermodynamic solubility for different crystalline and amorphous dihydrochloride salt forms have been disclosed at a concentration of 30 mg/ml in aqueous system, there is no data given with commercial viability of trilaciclib in the aqueous system that can be directly administered to the patient.

Goruva, et al., wjpmr, 2022, 8 (11), 141-153 discloses non-aqueous formulations comprising N, N dimethylacetamide, tocopherol, polysorbate 80, polyethylene glycol 400 and acetic acid. Further, discloses that terminal sterilization with autoclaving is not feasible for Trilaciclib Injection 10 mg/mL which led to the decreasing concentration of trilaciclib and increasing the formation of impurity levels post autoclave [Table 23-pages 150-151 of Goruva, et al.].

PCT Patent No. WO 2024/009319 discloses liquid injectable compositions of trilaciclib comprising of aqueous solvents or non-aqueous solvents suitable for injection. This application provided stability for non-aqueous formulations up to three months only which is not commercially viable and there is no significance disclosure given to towards stable aqueous formulation.

Aqueous injections generally offer advantages like ease of formulation and physiological compatibility compared to non-aqueous injections. Aqueous injections generally well tolerated as water is a natural component of body and less likely to cause irritation or inflammation at the injection site while non-aqueous vehicles can cause pain and irritation at the injection site. In the above mentioned literature, due to limited aqueous solubility of trilaciclib, there is no robust method disclosed for development for stable aqueous compositions. Accordingly, there exists a need to develop stable, aqueous compositions of trilaciclib which are easy to manufacture and provide improved patient's compliance.

### SUMMARY OF INVENTION

The inventors of the present invention surprisingly found that heating of the aqueous composition of trilaciclib for a specified time period produces a stable composition that does not precipitate at room temperature.

The present invention relates to an aqueous compositions comprising trilaciclib and solvent system suitable for injection. In particular, the invention discloses room temperature stable trilaciclib composition comprising trilaciclib, optionally a stabilizer wherein the compositions are stable at room temperature for at least one month or at least 3 months or at least 6 months or at least one year at least two years.

The present invention relates to an aqueous compositions comprising trilaciclib and solvent system suitable for injection, wherein the compositions are ready-to-dilute.

The present invention relates to a process for producing a stable aqueous compositions comprising trilaciclib and solvent system suitable for injection.

The invention accordingly provides a process for producing a stable aqueous pharmaceutical composition of trilaciclib suitable for parenteral administration, wherein the process comprises heating the composition from about 50° C. to 150° C. for 5 minutes to 4 hours to obtain a composition stable at room temperature for a period of at least 6 months.

The invention provides a process for producing a ready-to-dilute aqueous pharmaceutical trilaciclib composition suitable for parenteral administration, the process comprising: (i) addition of required quantity of water and mannitol to a manufacturing vessel and stirred to obtain a solution (ii) addition of required quantity of buffer to the solution obtained in step (i); and (iii) addition of required quantity of trilaciclib to the solution obtained in step (ii) followed by heating the composition from about 50° C. to 150° C. for 5 minutes to 4 hours to obtain a stable pharmaceutical composition of trilaciclib.

Further, the disclosed trilaciclib formulations/compositions of the invention are stable at room temperature and maintains at least 98% of trilaciclib content and/or contains less than 2% of total impurities as determined by HPLC, after storage at room temperature for a period of at least 6 months.

### DETAILED DESCRIPTION OF THE INVENTION

The present invention discloses an aqueous compositions comprising trilaciclib or its pharmaceutically acceptable salts, hydrates, solvates, polymorphs, and mixtures thereof and solvent system suitable for injection. Trilaciclib is prone for degradation in aqueous solutions, particularly when subjected to high temperature and humidity conditions, especially due to which numerous organic impurities, and inorganic impurities may be present in the solutions. The presence of such impurities tends to affect the long-term stability of the composition. The liquid compositions of trilaciclib are not stable at room temperature. While doing attempts to improve the stability at room temperature, the inventors of the present invention found that, the temperature impacts stability of trilaciclib. When heating performed on the bulk solution or vials of the trilaciclib for a period ranging from about 50° C. to 150° C. for 5 minutes to 4 hours the obtained pharmaceutical composition is stable for a period of at least 6 months with total impurities to be less than 2% based on total weight of composition.

The present invention discloses an aqueous compositions comprising trilaciclib or its pharmaceutically acceptable salts, hydrates, solvates, polymorphs, and mixtures thereof and solvent system suitable for injection, wherein the compositions are ready-to-dilute.

As used herein the term “ready-to-dilute” refers to a formulation which can be directly combined with a diluent (e.g., dextrose solution, saline solution, or any other infusion medium) and then administered to a patient. A sufficient amount of a concentrated, ready to dilute liquid formulation of trilaciclib admixed with a suitable fixed volume diluent container such as a bag containing 50, 100, 250 ml of 0.9% sodium chloride injection or 5% dextrose Injection. Further, “ready-to-dilute” formulation avoids a reconstitution step compared to lyophilized powder formulations

As used herein the term “ready-to-use” refers to any preparation of trilaciclib which can be administered to patient directly without any further dilution or processing.

As used herein, the terms “composition” and “formulation” refer to preparations comprising trilaciclib or its pharmaceutically acceptable salts, hydrates, solvates, polymorphs, and mixtures thereof in a form suitable for administration to a human of a mammal. The terms composition or formulation are used interchangeably in the present invention.

The “solvent system” refers to mixture of one or more solvents in a definite proportion which is suitable for dissolving the active ingredient. The solvent system in the present invention includes aqueous solvents include but not limited to purified water, dextrose solution, saline solution.

As used herein, the term “stable” refers to physical and/or chemical stability for commercially significant periods, such as at least 1 week or 1 month or 3 months or 6 months or 1 year or 2 years or 3 years, without significant physical instability (description, clarity etc.) and chemical degradation. Stable or stability may represent stability when stored at 2° C.-8° C. or at ambient conditions (e.g., 25° C.) or elevated temperatures (e.g., 40° C.).

As used herein the term “bulk solution” refers to a final composition which contains all the excipients before sterilization. The bulk solution refers to a composition comprising trilaciclib, organic acid, stabilizer and, optionally pH adjusting agents.

As used herein the term “organic acid” refers to a weak acid contains at least one carbon atom in its molecular framework. According to the invention organic acids refers to but not limited to aspartic acid, acetic acid, citric acid, lactic acid and tartaric acid.

As used herein, the term “heat treatment” refers to any source which increase the temperature of the trilaciclib composition. Heat treatment may be applied to various stages of manufacturing process and preferably applied for the bulk solution or after filling bulk solution into vials.

The term “pharmaceutically acceptable” refers to ingredients that are useful for preparing pharmaceutical compositions, and that is considered to be generally safe, non-toxic and neither biologically nor otherwise undesirable, and includes those ingredients acceptable for veterinary use as well as human pharmaceutical use.

As used herein, the term “pharmaceutically acceptable excipients” refers to pH adjusting agent, antioxidants, diluents, surfactants, suspending agents, emulsifying agents and combinations thereof.

The term “trilaciclib” is intended to include any of the alternative forms in which trilaciclib can be administered such as salts, esters, anhydrous, hydrates such as monohydrate or dihydrate, solvates, crystalline or amorphous polymorphs, racemic mixtures, enantiomeric isomers and so on.

The present invention discloses a process for producing an aqueous compositions comprising trilaciclib and solvent system suitable for injection.

In an embodiment, the present invention discloses an aqueous composition comprising about 0.1 mg/mL to about 25 mg/mL of trilaciclib or its pharmaceutically acceptable salts, hydrates, solvates, polymorphs, and mixtures thereof, preferably from about 0.5 mg/mL to about 15 mg/mL of trilaciclib or its pharmaceutically acceptable salts, hydrates, solvates, polymorphs, and mixtures thereof.

In the above mentioned embodiment, the composition of trilaciclib may further comprises buffering agents.

A buffer is present as an acid or a base and its conjugate base or acid, respectively. According to the present disclosure the buffering agent may comprise at least one buffering agent selected from the group consisting of citrate (sodium or potassium), sodium acetate, phosphate, malate, lactate, tromethamine (Tris) and mixtures thereof. The most used acids are citric acid, tartaric acid and aspartic acid. The amount of buffering agent used differs based on the buffering agent used in the composition. The buffering agent may be present in an amount of about 0.01 mg/mL to about 100 mg/mL.

In another embodiment, the disclosed formulations of trilaciclib further comprises stabilizers.

The term “stabilizer” identifies an agent which improves the composition physical and chemical stability for commercially significant periods, such as at least 1 week, 1 month, 2 months, 3 months, 6 months, 1 year, 2 years, 3 years, without significant physical instability (description, clarity etc.) and chemical degradation. Stable or stability may represent stability when stored at 2° C.-8° C. or at ambient conditions (e.g. 25° C.) or elevated temperatures (e.g. 40° C.). Suitable stabilizers include but are not limited to sucrose, mannitol, citric acid, tartaric acid and aspartic acid, cyclodextrin include hydroxypropyl beta-cyclodextrin (HPBCD) and sulfobutylether beta-cyclodextrin (SBECD) and combination thereof. The stabilizer is present in the amount of about 5 mg/mL to about 100 mg/mL.

Below are the impurities for trilaciclib known from the literature

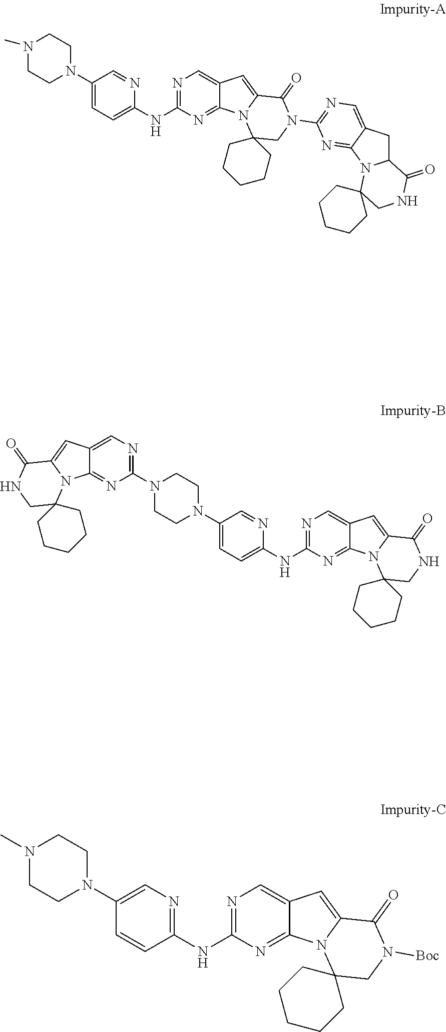 [SMILES: CN1CCN(c2ccc(Nc3ncc4cc5n(c4n3)C3(CCCCC3)CN(c3ncc4c(n3)N3C(C4)C(=O)NCC34CCCCC4)C5=O)nc2)CC1 ; O=C1NCC2(CCCCC2)n2c1cc1cnc(Nc3ccc(N4CCN(c5ncc6cc7n(c6n5)C5(CCCCC5)CNC7=O)CC4)cn3)nc12 ; CN1CCN(c2ccc(Nc3ncc4cc5n(c4n3)C3(CCCCC3)CN(C(=O)OC(C)(C)C)C5=O)nc2)CC1]

In an embodiment the invention discloses a stable liquid formulation of trilaciclib herein levels of impurity A in the formulation is less than about 0.2% by weight of label content of trilaciclib or its salt when stored at 25° C. temperature for 6 months at 60% relative humidity (RH).

In an embodiment the invention discloses a stable liquid formulation of trilaciclib herein levels of impurity A in the formulation is less than about 0.2% by weight of label content of trilaciclib or its salt when stored at 40° C. temperature for 6 months at 75% RH.

In an embodiment the invention discloses a stable liquid formulation of trilaciclib herein levels of impurity B in the formulation is less than about 0.2% by weight of label content of trilaciclib or its salt when stored at 25° C. temperature for 6 months at 60% relative humidity (RH).

In an embodiment the invention discloses a stable liquid formulation of trilaciclib herein levels of impurity B in the formulation is less than about 0.2% by weight of label content of trilaciclib or its salt when stored at 40° C. temperature for 6 months at 75% RH.

In an embodiment the invention discloses a stable liquid formulation of trilaciclib herein levels of impurity C in the formulation is less than about 0.2% by weight of label content of trilaciclib or its salt when stored at 25° C. temperature for 6 months at 60% relative humidity (RH).

In an embodiment the invention discloses a stable liquid formulation of trilaciclib herein levels of impurity C in the formulation is less than about 0.2% by weight of label content of trilaciclib or its salt when stored at 40° C. temperature for 6 months at 75% RH.

In yet another embodiment, the disclosed formulations of trilaciclib further comprises pH adjusting agents such as sodium hydroxide or hydrochloric acid to adjust the pH as desired.

In the present invention, it has been also found that when liquid compositions of trilaciclib in vials filled with non-oxygen gas result in significantly enhanced room-temperature stability whereas vials filled without any non-oxygen gas significantly impact the stability and are not suitable for room temperature storage as desired. The non-oxygen containing gas is selected from nitrogen, argon, helium, and neon. Nitrogen is the preferred non-oxygen containing gas.

In one embodiment, the present invention discloses an aqueous pharmaceutical composition comprising:
a) trilaciclib or a pharmaceutically acceptable salt thereof,
b) an organic acid,
c) stabilizing agent, and
d) optionally, one or more pharmaceutically acceptable excipients, wherein the composition has a pH between 2.0 to 6.0.

In another embodiment, the present invention discloses an aqueous pharmaceutical composition comprising:
a) trilaciclib or a pharmaceutically acceptable salt thereof,
b) an organic acid selected from the group consisting of citric acid or tartaric acid or aspartic acid,
c) stabilizing agent, and
d) optionally, one or more pharmaceutically acceptable excipients,
wherein the composition has a pH between 2.0 to 6.0.

In one embodiment, the present invention discloses an aqueous pharmaceutical composition comprising:
a) 0.1 mg/mL to 25 mg/mL of trilaciclib or a pharmaceutically acceptable salt thereof,
b) citric acid,
c) mannitol, and
d) optionally, one or more pharmaceutically acceptable excipients,
wherein the composition has a pH between 2.0 to 6.0.

In another embodiment, the present invention discloses a room temperature stable aqueous pharmaceutical composition comprising:
a) 0.1 mg/mL to 25 mg/mL of trilaciclib or a pharmaceutically acceptable salt thereof,
b) citric acid,
c) mannitol, and
d) optionally, one or more pharmaceutically acceptable excipients,
wherein the composition has a pH between 2.0 to 6.0.

In yet another embodiment, the present invention discloses an aqueous pharmaceutical composition comprising:
a) 0.1 mg/mL to 25 mg/mL of trilaciclib or a pharmaceutically acceptable salt thereof,
b) tartaric acid,
c) mannitol, and
d) optionally, one or more other pharmaceutically acceptable excipients,
wherein the composition has a pH between 2.0 to 6.0.

In another embodiment, the present invention discloses a room temperature stable aqueous pharmaceutical composition comprising:
a) 0.1 mg/mL to 25 mg/mL of trilaciclib or a pharmaceutically acceptable salt thereof,
b) tartaric acid,
c) mannitol, and
d) optionally, one or more other pharmaceutically acceptable excipients,
wherein the composition has a pH between 2.0 to 6.0.

In one embodiment, the present invention discloses aqueous pharmaceutical composition comprising:
a) 0.1 mg/mL to 25 mg/mL of trilaciclib or a pharmaceutically acceptable salt thereof,
b) aspartic acid,
c) mannitol, and
d) optionally, one or more other pharmaceutically acceptable excipients,
wherein the composition has a pH between 2.0 to 6.0.

In another embodiment, the present invention discloses a room temperature stable aqueous pharmaceutical composition comprising:
a) 0.1 mg/mL to 25 mg/mL of trilaciclib or a pharmaceutically acceptable salt thereof,
b) aspartic acid,
c) mannitol, and
d) optionally, one or more other pharmaceutically acceptable excipients,
wherein the composition has a pH between 2.0 to 6.0.

In one embodiment, the present invention discloses aqueous pharmaceutical composition comprising:
a) about 300 mg-500 mg of trilaciclib or a pharmaceutically acceptable salt thereof,
b) about 50 mg-100 mg citric acid,
c) about 250-350 mg mannitol, and
d) optionally, sodium hydroxide and/or hydrochloric acid to adjust the pH as desired,
wherein the composition is prepared by a process which includes a step of heating the composition from about 50° C. to 150° C. for 5 minutes to 4 hours.

In one embodiment, the present invention discloses an aqueous ready-to-dilute pharmaceutical composition comprising:
a) about 300 mg of trilaciclib or a pharmaceutically acceptable salt thereof,
b) about 76 mg citric acid,
c) about 300 mg mannitol, and
d) optionally, sodium hydroxide and/or hydrochloric acid to adjust the pH as desired,
wherein the composition is prepared by a process which includes a step of heating the composition from about 110° C. to 125° C. for 5 minutes to 30 minutes.

In one embodiment, the present invention discloses to aqueous pharmaceutical composition comprising:
a) about 300 mg-500 mg of trilaciclib or a pharmaceutically acceptable salt thereof,
b) about 5 mg-50 mg tartaric acid,
c) about 250 mg-350 mg mannitol, and
d) optionally, sodium hydroxide and/or hydrochloric acid to adjust the pH as desired,
wherein the composition is prepared by a process a heating the composition from about 50° C. to 150° C. for 5 minutes to 4 hours.

In one embodiment, the present invention discloses an aqueous ready-to-dilute pharmaceutical composition comprising:
a) about 300 mg of trilaciclib or a pharmaceutically acceptable salt thereof,
b) about 32 mg tartaric acid,
c) about 300 mg mannitol, and
d) optionally, sodium hydroxide and/or hydrochloric acid to adjust the pH as desired,
wherein the composition is prepared by a process which includes a step of heating the composition from about 110° C. to 125° C. for 5 minutes to 30 minutes.

In one embodiment, the present invention discloses an aqueous pharmaceutical composition comprising:
a) about 300 mg-500 mg of trilaciclib or a pharmaceutically acceptable salt thereof,
b) about 5 mg-50 mg aspartic acid,
c) about 250 mg-350 mg mannitol, and
d) optionally, sodium hydroxide and/or hydrochloric acid to adjust the pH as desired,
wherein the composition is prepared by a process which includes a step of heating the composition from about 50° C. to 150° C. for 5 minutes to 4 hours.

In one embodiment, the present invention relates to aqueous ready-to-dilute pharmaceutical composition comprising:
a) about 300 mg of trilaciclib or a pharmaceutically acceptable salt thereof,
b) about 14 mg aspartic acid,
c) about 300 mg mannitol, and
d) optionally, sodium hydroxide and/or hydrochloric acid to adjust the pH as desired,
wherein the composition is prepared by a process which includes a step of heating the composition from about 110° C. to 125° C. for 5 minutes to 30 minutes.

In another embodiment this invention discloses a method for preparing a sterile aqueous pharmaceutical composition, the method comprising:
(a) combining trilaciclib with pharmaceutically acceptable excipients to form a bulk solution; and
(b) heating the bulk solution to a temperature from about 50° C. to 150° C. for a period of 5 minutes to 4 hours to yield the sterile aqueous pharmaceutical composition.

In another embodiment this invention discloses a process to prepare the ready-to-dilute stable parenteral composition of trilaciclib wherein the process comprising:
a) addition required quantity of water in a manufacturing vessel under nitrogen purging,
b) addition of mannitol to the above vessel and stir to obtain a clear solution,
c) addition of organic acid selected from the group consisting of citric acid or tartaric acid or aspartic acid to the clear solution obtained in step b) and stir further to obtain a clear solution, followed by
d) addition of trilaciclib to the solution obtained in step c) and stir to obtain a clear solution, followed by
e) adjusting the pH of the solution obtained in step d) using suitable acidifying agent.
f) filtering the solution and filling the filtered solution into vials and subjecting the vials to heating from about 110° C. to 125° C. for about 5 minutes to 4 hours.

In another embodiment this invention discloses a process to prepare the ready-to-dilute stable parenteral composition of trilaciclib wherein the process comprising:
a) addition required quantity of water in a manufacturing vessel under nitrogen purging,
b) addition of mannitol to the above vessel and stir to obtain a clear solution,
c) addition of organic acid selected from the group consisting of citric acid or tartaric acid or aspartic acid to the clear solution obtained in step b) and stir further to obtain a clear solution, followed by
d) addition of trilaciclib to the solution obtained in step c) and stir to obtain a clear solution, followed by
e) adjusting the pH of the solution obtained in step d) using suitable acidifying agent.
f) filtering the solution and subjecting the above bulk solution of step e) to heating from about 110° C. to 125° C. for about 5 minutes to 4 hours.

In another embodiment this invention discloses a process to prepare the ready-to-dilute stable parenteral composition of trilaciclib wherein the process comprising:
a) addition required quantity of water in a manufacturing vessel under nitrogen purging,
b) addition of mannitol to the above vessel and stir to obtain a clear solution,
c) addition of citric acid to the clear solution obtained in step b) and stir further to obtain a clear solution, followed by
d) addition of trilaciclib to the solution obtained in step c) and stir to obtain a clear solution, followed by
e) adjusting the pH of the solution obtained in step d) using suitable acidifying agent.

In the above-mentioned embodiment, the process further comprises a step of filtration and filling the filtered solution into vials and subjected to autoclaving at about 121° C. to 124° C. for about 5 minutes to 30 minutes.

In another embodiment this invention discloses a process to prepare a ready-to-dilute stable parenteral composition of trilaciclib wherein the process comprising:
a) addition required quantity of water in a manufacturing vessel under nitrogen purging,
b) addition of mannitol to the above vessel and stir to obtain a clear solution,
c) addition of aspartic acid to the clear solution obtained in step b) and stir further to obtain a clear solution, followed by
d) addition of trilaciclib to the solution obtained in step c) and stir to obtain a clear solution, followed by
e) adjusting the pH of the solution obtained in step d) using suitable acidifying agent.

In the above-mentioned embodiment, the process further comprises a step of filtration and filling the filtered solution into vials and subjected to autoclaving at about 121° C. to 124° C. for about 5 minutes to 30 minutes.

In another embodiment this invention discloses a process to prepare the ready-to-dilute stable parenteral composition of trilaciclib wherein the process comprising:
a) addition required quantity of water in a manufacturing vessel under nitrogen purging,
b) addition of mannitol to the above vessel and stir to obtain a clear solution,
c) addition of tartaric acid to the clear solution obtained in step b) and stir further to obtain a clear solution, followed by
d) addition of trilaciclib to the solution obtained in step c) and stir to obtain a clear solution, followed by
e) adjusting the pH of the solution obtained in step d) using suitable acidifying agent.

In the above-mentioned embodiment, the process further comprises a step of filtration and filling the filtered solution into vials and subjected to autoclaving at about 121° C. to 124° C. for about 5 minutes to 30 minutes.

In the above-mentioned embodiment, the process of terminal heat treatment stabilizes the composition and decreases the total impurity levels to less than 2% when stored at room temperature for at least one month, or at least 3 months, or at least six months.

In the above mentioned embodiment, the process of terminal heat treatment stabilizes the composition and decreases the total impurity levels to less than 1% when stored at room temperature for at least one month, or at least 3 months, or at least six months.

In some embodiments, the liquid pharmaceutical composition disclosed herein do not exhibit substantial degradation under room temperature for at least 3 months, at least 6 months, at least 8 months, at least 10 months, at least 12 months, or at least 24 months.

Injectable formulations can be administered through various routes such as intramuscular, intra-peritoneal, intravenous or subcutaneous administration. Preferred routes of administration are subcutaneous or intravenous route of administration for the composition of the present invention.

According to a preferred embodiment, the present invention discloses a liquid injectable composition comprising trilaciclib; wherein the injectable composition is packaged in a container suitable for both single and multi-use. The preferred containers include an ampoule, a vial, a pre-filled syringe, and intravenous bag.

The following examples further describe certain specific formulations of the invention and demonstrate the practice and advantages thereof. A general manufacturing process is given for Examples. It is to be understood that the examples and manufacturing are given by way of illustration only and are not intended to limit the scope of the invention in any manner.

### EXAMPLES

### Example-1

TABLE 1
Manufacturing formula

| S. No. | Formulation | 15 mg/mL | | qtty/Vial |
|---|---|---|---|---|
| 1 | Trilaciclib Dihydrochloride | 17.45 | mg/mL | 349 mg |
| 2 | Citric acid | 3.78 | mg | 75.6 mg |
| 3 | Mannitol | 15 | mg/mL | 300 mg |

| | | | |
|---|---|---|---|
| 4 | Hydrochloric acid | q.s. to | q.s. to |
| | | adjust pH | adjust pH |
| 5 | Sodium hydroxide | q.s. to | q.s. to |
| | | adjust pH | adjust pH |
| 7 | Water for injection | q.s. to | q.s. to |
| | | 1 mL | 20 mL |

### Manufacturing Process

The required quantity of water for injection taken to the manufacturing vessel and nitrogen is purged into the manufacturing vessel to ensure that the dissolved oxygen should be less than 2 ppm. Mannitol and citric acid were added to this and stirred to obtain a clear solution. The required quantity of trilaciclib was added to this solution and stirred to obtain a clear solution. pH of this solution adjusted to 4 using 1N hydrochloric acid/sodium hydroxide. Water for injection was added to make up the volume. The solution was filtered through a 0.22 micron PVDF (polyvinylidene difluoride) filter. The solution was filled in glass vials and subjected to heat treatment at 121° C. for about 8 minutes to 30 minutes. Further, the samples were stored at room temperature for six months and stability of these samples were measured using HPLC technique. Results of the study is given below.

TABLE 2
Stability data of Example-1 (With headspace nitrogen)

| | Condition→ | | | | | | |
|---|---|---|---|---|---|---|---|
| | | 40 °C/75% RH | | | | 25 °C/60% RH | |
| | Limit | Initial | 1 M | 3 M | 6 M | 3 M | 6 M |
| pH | 3.5 to 4.3 | 4.11 | 3.63 | 4.02 | 4.05 | 4.01 | 4.05 |
| Assay | 90% to 110% | 101.7 | 102.0 | 102.8 | 100.4 | 102.7 | 101.6 |

Related substances (%)

| | | | | | | | |
|---|---|---|---|---|---|---|---|
| Single Unknown max | 0.2% | BDL | BDL | BDL | 0.05 | BDL | 0.102 |
| Total impurities | NMT 1.5% | 0.11 | 0.08 | 0.13 | 0.22 | 0.17 | 0.20 |
| Particulate matter | 10 μ - NMT 6000 | 172.0 | 189.3 | 22.0 | 10.0 | 40.0 | 39.0 |
| | 25 μ - NMT 600 | 13.0 | 8.00 | 0.00 | 0.00 | 3.00 | 4.00 |

BDL: Below detection limit;
NMT—Not more than

TABLE 3
Stability data of Example-1 (Without headspace nitrogen)

| | Condition→ | | | | | | |
|---|---|---|---|---|---|---|---|
| | | 40 °C/75% RH | | | | 25 °C/60% RH | |
| | Limit | Initial | 1 M | 3 M | 6 M | 3 M | 6 M |
| pH | 3.5 to 4.3 | 4.09 | 4.12 | 4.14 | 4.04 | 4.14 | 4.03 |
| Assay | 90% to 110% | 96.65 | 97.86 | 100.6 | 100.4 | 101.5 | 101.9 |

Related substances (%)

| | | | | | | | |
|---|---|---|---|---|---|---|---|
| Single Unknown max | 0.2% | 0.06 | 0.10 | 0.20 | 0.21 | 0.05 | 0.09 |
| Total impurities | NMT 1.5% | 0.06 | 0.41 | 0.79 | 1.08 | 0.19 | 0.35 |
| Particulate matter | 10 μ - NMT 6000 | 3030 | 160 | 170.6 | 144 | 138.6 | 101.3 |
| | 25 μ - NMT 600 | 30 | 12 | 8 | 6.67 | 6.67 | 1.33 |

NMT—Not more than

### Stability Data of Compositions of Example-1 Under Various Temperature Conditions for a Different Time Period

TABLE 4
Composition autoclaved 121 °C for 8 minutes

| | Condition→ | | | | | | |
|---|---|---|---|---|---|---|---|
| | | 40 °C/75% RH | | | | 25 °C/60% RH | |
| | Limit | Initial | 1 M | 3 M | 6 M | 3 M | 6 M |
| pH | 3.5 to 4.3 | 4.03 | 3.96 | 3.97 | 3.94 | 3.98 | 3.87 |
| Assay | 90% to 110% | 102.0 | 102.8 | 102.4 | 102.1 | 103.8 | 100.9 |

Related substances (%)

| | | | | | | | |
|---|---|---|---|---|---|---|---|
| Single Unknown max | 0.2% | BDL | BDL | 0.051 | 0.065 | 0.068 | 0.073 |
| Total impurities | NMT 1.5% | 0.11 | 0.08 | 0.23 | 0.25 | 0.22 | 0.21 |
| Particulate matter | 10 μ - NMT 6000 | 41 | 26 | 22 | 91 | 40 | 248 |
| | 25 μ - NMT 600 | 2 | 0 | 0 | 3 | 3 | 16 |

BDL: Below detection limit;
NMT—Not more than

TABLE 5
Composition autoclaved 121 °C for 15 minutes

| | Condition→ | | | | | | |
|---|---|---|---|---|---|---|---|
| | | 40 °C/75% RH | | | | 25 °C/60% RH | |
| | Limit | Initial | 1 M | 3 M | 6 M | 3 M | 6 M |
| pH | 3.5 to 4.3 | 4.03 | 3.96 | 3.97 | 3.90 | 3.96 | 3.95 |
| Assay | 90% to110% | 104.1 | 101.2 | 101.4 | 102.2 | 101.6 | 99.8 |

Related substances (%)

| | | | | | | | |
|---|---|---|---|---|---|---|---|
| Single Unknown max | 0.2% | BDL | BDL | BDL | 0.10 | BDL | 0.07 |
| Total impurities | NMT 1.5% | 0.11 | 0.08 | 0.22 | 0.26 | 0.16 | 0.18 |
| Particulate matter | 10 μ - NMT 6000 | 24 | 92 | 186 | 150.6 | 25 | 505 |
| | 25 μ - NMT 600 | 2 | 12 | 8 | 1.33 | 3 | 9 |

BDL: Below detection limit;
NMT—Not more than

TABLE 6
Composition on Hold for 6 Hours at 70 °C

| | Analytical | | | 3 | 6 |
|---|---|---|---|---|---|
| | tests | Specification | Initial | Hrs. | Hrs. |
| Container | Related substances (%) | | | | |
| Glass | Any unspecified | NMT 0.2% | 0.0179 | 0.0214 | 0.0265 |
| container | Degradation | | | | |
| | product | | | | |
| | Total degradation | NMT 1.0% | 0.0537 | 0.0596 | 0.0751 |
| | products | | | | |

NMT—Not more than

TABLE 7
Composition on Hold for 6 Hours at 60 °C

| | Analytical | | | 3 | 6 |
|---|---|---|---|---|---|
| | tests | Specification | Initial | Hrs. | Hrs. |
| Container | Related substances (%) | | | | |
| Glass | Any unspecified | NMT 0.2% | 0.037 | 0.081 | 0.090 |
| container | Degradation | | | | |
| | product | | | | |
| | Total degradation | NMT 1.0% | 0.126 | 0.228 | 0.245 |
| | products | | | | |

NMT—Not more than

TABLE 8
Dilution Study data:

| | | | | Particulate matter | | |
|---|---|---|---|---|---|---|
| | Concentration | | Osmolality | 10 μ (NMT | 25 μ (NMT | |
| Diluent | (mg/ml) | pH | (mOsmol/kg) | 6000/cont.) | 600/cont.) | Remarks |
| 0.9% | 3 | 4.34 | 264 | 546.67 | 33.33 | Stable up to |
| NaCl | 0.5 | 4.55 | 279 | 660.00 | 6.67 | 8 hours |
| 5% | 3 | 4.31 | 251 | 192.00 | 6.00 | Stable up to |
| Dextrose | 0.5 | 4.65 | 261 | 63.00 | 0.00 | 12 hours |

### Example-2

TABLE 9
Manufacturing formula

| S. No. | Formulation | 15 mg/mL | qtty/Vial |
|---|---|---|---|
| 1 | Trilaciclib Dihydrochloride | 17.45 mg | 349 mg |
| 2 | Tartaric acid | 1.6 mg | 32.0 mg |
| 3 | Mannitol | 15 mg | 300 mg |
| 4 | Hydrochloric acid | q.s. to | q.s. to |
| | | adjust pH | adjust pH |
| 5 | Sodium hydroxide | q.s. to | q.s. to |
| | | adjust pH | adjust pH |
| 7 | Water for injection | q.s. to | q.s. to |
| | | 1 mL | 20 mL |

### Manufacturing Process

The required quantity of water for injection taken to the manufacturing vessel and nitrogen is purged into the manufacturing vessel to ensure that the dissolved oxygen should be less than 2 ppm. Mannitol and tartaric acid were added to this and stirred to obtain a clear solution. The required quantity of trilaciclib was added to this solution and stirred to obtain a clear solution. pH of this solution adjusted to 4 using 1N hydrochloric acid/sodium hydroxide. Water for injection was added to make up the volume. The solution was filtered through a 0.22 micron PVDF (polyvinylidene difluoride) filter. The solution was filled in glass vials and subjected to heat treatment at 121° C. for about 8 minutes to 30 minutes. Further, the samples were stored at room temperature for six months and stability of these samples were measured using HPLC technique. Results of the study is given below.

TABLE 10
Stability data of Example 2 (With headspace nitrogen)

| | Condition→ | | | | | | |
|---|---|---|---|---|---|---|---|
| | | 40 °C/75% RH | | | | 25 °C/60% RH | |
| | Limit | Initial | 1 M | 3 M | 6 M | 3 M | 6 M |
| pH | 3.5 to 4.3 | 3.99 | 3.97 | 3.96 | 4.03 | 3.95 | 4.02 |
| Assay | 90% to 110% | 102.7 | 101.4 | 104.2 | 103.4 | 104.0 | 103.1 |

Related substances (%)

| | | | | | | | |
|---|---|---|---|---|---|---|---|
| Single Unknown max | 0.2% | BDL | BDL | 0.06 | 0.05 | 0.05 | 0.06 |
| Total impurities | NMT 1.5% | 0.06 | 0.18 | 0.25 | 0.26 | 0.23 | 0.19 |
| Particulate matter | 10 μ - NMT 6000 | — | — | 25.3 | 64.0 | 20.0 | 232 |
| | 25 μ - NMT 600 | — | — | 2.67 | 5.33 | 0.00 | 8 |

BDL: Below detection limit;
NMT—Not more than

### Example-3

TABLE 11
Manufacturing formula

| S. No. | Formulation | 15 mg/mL | qtty/Vial |
|---|---|---|---|
| 1 | Trilaciclib Dihydrochloride | 17.45 mg | 349 mg |
| 2 | Aspartic acid | 0.7 mg | 14.0 mg |
| 3 | Mannitol | 15 mg | 300 mg |
| 4 | Hydrochloric acid | q.s. to | q.s. to |
| | | adjust pH | adjust pH |
| 5 | Sodium hydroxide | q.s. to | q.s. to |
| | | adjust pH | adjust pH |
| 7 | Water for injection | q.s. to | q.s. to |
| | | 1 mL | 20 mL |

### Manufacturing Process

The required quantity of water for injection taken to the manufacturing vessel and nitrogen is purged into the manufacturing vessel to ensure that the dissolved oxygen should be less than 2 ppm. Mannitol and Aspartic acid were added to this and stirred to obtain a clear solution. The required quantity of trilaciclib was added to this solution and stirred to obtain a clear solution. pH of this solution adjusted to 4 using 1N hydrochloric acid/sodium hydroxide. Water for injection was added to make up the volume. The solution was filtered through a 0.22 micron PVDF (polyvinylidene difluoride) filter. The solution was filled in glass vials and subjected to heat treatment at 121° C. for about 8 minutes to 30 minutes. Further, the samples were stored at room temperature for six months and stability of these samples were measured using HPLC technique. Results of the study is given below.

TABLE 12
Stability data of Example-3 (With headspace nitrogen)

| | Condition→ | | | | | | |
|---|---|---|---|---|---|---|---|
| | | 40 °C/75% RH | | | | 25 °C/60% RH | |
| | Limit | Initial | 1 M | 3 M | 6 M | 3 M | 6 M |
| pH | 3.5 to 4.3 | 3.68 | 3.62 | 3.98 | 4.03 | 3.95 | 4.02 |
| Assay | 90% to 110% | 101.7 | 102.61 | 104.5 | 103.3 | 103.5 | 103.9 |

Related substances (%)

| | | | | | | | |
|---|---|---|---|---|---|---|---|
| Single Unknown max | 0.2% | BDL | BDL | 0.06 | 0.05 | 0.06 | 0.05 |
| Total impurities | NMT 1.5% | 0.06 | 0.15 | 0.22 | 0.27 | 0.20 | 0.18 |
| Particulate matter | 10 μ - NMT 6000 | — | — | 1453.3 | 337.3 | 1290.6 | 1693.3 |
| | 25 μ - NMT 600 | — | — | 42.67 | 18.67 | 42.67 | 66.67 |

We claim:

1. An aqueous pharmaceutical composition comprising:
a) trilaciclib or a pharmaceutically acceptable salt thereof,
b) an organic acid selected from citric acid or tartaric acid or aspartic acid, and
c) mannitol,
wherein the composition is prepared by a process comprising a step of heating the composition at a temperature ranging from about 50° C. to 150° C. for a duration of about 5 minutes to about 4 hours.

2. The pharmaceutical composition according to claim 1, wherein the composition is a ready-to-dilute composition.

3. The pharmaceutical composition according to claim 1, further comprising a pH adjusting agent selected from sodium hydroxide or hydrochloric acid or combination thereof.

4. The pharmaceutical composition according to claim 1, wherein the composition has a pH between 2.0 to 6.0.

5. The pharmaceutical composition according to claim 1, wherein the composition is stable at room temperature for at least one month or at least 3 months or at least 6 months or at least one year or at least two years.

6. An aqueous ready-to-dilute pharmaceutical composition comprising:
a) about 300 mg of trilaciclib or a pharmaceutically acceptable salt thereof,
b) about 76 mg citric acid or about 32 mg tartaric acid or about 14 mg aspartic acid,
c) about 300 mg mannitol, and
d) optionally, a pH adjusting agent selected from sodium hydroxide or hydrochloric acid or combination thereof,
wherein the composition is prepared by a process comprising a step of heating the composition at a temperature ranging from about 50° C. to 150° C. for a duration of about 5 minutes to about 4 hours.

7. The pharmaceutical composition according to claim 6, wherein the composition consists of:
a) about 300 mg of trilaciclib or a pharmaceutically acceptable salt thereof,
b) about 76 mg citric acid,
c) about 300 mg mannitol, and
d) optionally, a pH adjusting agent selected from sodium hydroxide or hydrochloric acid or combination thereof,
wherein the composition is prepared by heating at a temperature ranging from about 110° C. to about 125° C. for about 5 minutes to about 30 minutes.

8. The pharmaceutical composition according to claim 6, wherein the composition consists of:
a) about 300 mg of trilaciclib or a pharmaceutically acceptable salt thereof,
b) about 32 mg tartaric acid,
c) about 300 mg mannitol, and
d) optionally, a pH-adjusting agent selected from sodium hydroxide or hydrochloric acid, or a combination thereof;
wherein the composition is prepared by heating at a temperature ranging from about 110° C. to 125° C. for 5 minutes to 30 minutes.

9. The pharmaceutical composition according to claim 6, wherein the composition consists of:
a) about 300 mg of trilaciclib or a pharmaceutically acceptable salt thereof,
b) about 14 mg aspartic acid,
c) about 300 mg mannitol, and
d) optionally a pH adjusting agent selected from sodium hydroxide or hydrochloric acid, or a combination thereof;
wherein the composition is prepared by heating at a temperature ranging from about 110° C. to 125° C. for about 5 minutes to about 30 minutes.

10. The pharmaceutical composition according to claim 6, wherein the composition contains less than about 0.2% (w/w) of Impurity A or Impurity B or Impurity C as determined by HPLC, after storage at room temperature for a period of at least 6 months.

11. The pharmaceutical composition according to claim 6, wherein the composition contains less than 2% of total impurities as determined by HPLC, after storage at room temperature for a period of at least 6 months.

12. The pharmaceutical composition according to claim 6, wherein the composition contains less than 2% of total impurities as determined by HPLC, after storage at 40° C. for a period of at least 6 months.

13. The pharmaceutical composition according to claim 6, wherein the composition is further diluted with 0.9% NaCl or 5% Dextrose solution prior to administration.

14. A method for preparing a sterile aqueous pharmaceutical composition, the method comprising:
(a) combining trilaciclib with pharmaceutically acceptable excipients to form a bulk solution, and
(b) heating the bulk solution to a temperature from about 50° C. to 150° C. for a period of 5 minutes to 4 hours to yield the sterile aqueous pharmaceutical composition.

15. The method according to claim 14 further comprising steps of:
a) providing a manufacturing vessel containing water under a nitrogen purge;
b) adding mannitol to the manufacturing vessel and stirring until a clear solution is obtained;
c) adding an organic acid selected from citric acid or tartaric acid or aspartic acid to the clear solution obtained in step b) and stirring until a clear solution is obtained;
d) adding trilaciclib or pharmaceutically acceptable salt thereof to the clear solution obtained in step c) and stirring until a clear solution is obtained;
e) adjusting the pH above solution obtained in step d) using an acidifying agent; and
f) sterilizing the solution obtained in step e) by autoclaving at a temperature ranging from about 121° C. to about 124° C. for a period ranging from about 5 to 30 minutes.
